(19) United States
(12) Patent Application Publication
(10) Pub. No.: US 2020/0209230 A1
(43) Pub. Date: Jul. 2, 2020

(54) METHODS AND COMPOSITIONS RELATING TO SMALL MOLECULE ANALYTE ASSAYS

(71) Applicant: Arbor Assays LLC, Ann Arbor, MI (US)

(72) Inventor: Russell Hart, Chelsea, MI (US)

(21) Appl. No.: 16/814,296

(22) Filed: Mar. 10, 2020

Related U.S. Application Data
(62) Division of application No. 15/633,212, filed on Jun. 26, 2017.
(60) Provisional application No. 62/354,410, filed on Jun. 24, 2016.

(51) Int. Cl. G01N 33/543; G01N 33/53; G01N 33/58

(52) U.S. Cl. CPC G01N 33/54306; G01N 33/581; G01N 33/5308

(57) ABSTRACT

Kits and methods according to aspects of the present invention relate to the detection and quantitation of small molecule analytes including 8-hydroxyguanosine, 8-hydroxyguanosine, and 8-hydroxy-2′-deoxyguanosine.

### REFERENCE TO RELATED APPLICATIONS

This application is a divisional of U.S. patent application Ser. No. 15/633,212, filed Jun. 26, 2017, which claims priority to U.S. Provisional Patent Application Ser. No. 62/354,410, filed Jun. 24, 2016, the entire content of both of which is incorporated herein by reference.

### FIELD OF THE INVENTION

The present invention relates generally to methods for detecting a small molecule analyte in a sample. In specific aspects, the present invention relates to methods for detecting a small molecule analyte in a sample using a competitive binding assay format,

### BACKGROUND OF THE INVENTION

Standard immunological techniques are difficult to use with success in the detection of small molecule analytes, particularly in low concentrations.

It is particularly desirable to detect low concentrations (1 nmolar or less) of such small molecule analytes in many samples, but commercially available assays are not always dependable for this purpose. For example, some assay systems for detection of DNA damage oxidation products, one or more guanine derivatives is attached to a carrier protein, and the carrier protein/guanine derivative conjugate is then coated onto a solid phase material, such as a microtiter plate. The levels of guanine derivative are measured using an antibody that specifically binds to the guanine derivative. In order to measure the very low amounts of DNA damage oxidation products, extremely to concentrations of carrier protein/guanine derivative conjugate need to be bound to the solid phase material. However, the ability to coat the solid phase material at low concentrations of a carrier protein/guanine derivative conjugate causes difficulties since the carrier protein/guanine derivative conjugate binds non-specifically to many surfaces, including containers, stirring devices, tubing, etc.

As such, there is a continuing need for improved methods and kits for the detection and quantification of small molecule analytes, such as guanine derivatives and/or guanosine derivatives caused by the oxidation of DNA.

### SUMMARY OF THE INVENTION

Methods for detecting a small molecule analyte in a sample are provided according to aspects of the present invention which include providing a first component including a receptor, wherein the first component is immobilized on a solid phase support, and wherein the receptor does not specifically bind to the small molecule analyte; providing a second component including a ligand for said receptor, said ligand conjugated to a conjugated small molecule analyte; providing a third component including a detection molecule conjugated to a label, the detection molecule capable of specific binding with the small molecule analyte, wherein said third component does not specifically bind to the first component; contacting the second component and the immobilized first component under specific binding reaction conditions, whereby the second component is immobilized on the solid phase support by specific binding of the receptor and the ligand, producing an immobilized second component; contacting a fluid sample containing or suspected of containing the small molecule analyte and the immobilized second component with, the third component under specific binding reaction conditions, whereby a labeled immobilized complex is formed between the third component and the immobilized second component by the binding between the detection molecule and the ligand conjugated small molecule analyte, and whereby a labeled non-immobilized complex is formed between the third component and the small molecule analyte of the fluid sample by specific binding between the detection molecule and the small molecule analyte of the fluid sample; separating the immobilized complex from the non-immobilized complex; and detecting a signal from the labeled immobilized complex, the labeled non-immobilized complex, or both the labeled immobilized complex and the labeled non-immobilized complex, thereby detecting the small molecule analyte in the sample.

Methods for quantitating a small molecule analyte in a sample are provided according to aspects of the present invention which include providing a first component including a receptor, wherein the first component is immobilized on a solid phase support, and wherein the receptor does not specifically bind to the small molecule analyte; providing a second component including a ligand for said receptor, said ligand conjugated to a conjugated small molecule analyte; providing a third component including a detection molecule conjugated to a label, the detection molecule capable of specific binding with the small molecule analyte, wherein said third component does not specifically bind to the first component; contacting the second component and the immobilized first component under specific binding reaction conditions, whereby the second component is immobilized on the solid phase support by specific binding of the receptor and the ligand, producing an immobilized second component; contacting a fluid sample containing or suspected of containing the small molecule analyte and the immobilized second component with the third component under specific binding reaction conditions, whereby a labeled immobilized complex is formed between the third component and the immobilized second component by the binding between the detection molecule and the conjugated small molecule analyte, and whereby a labeled non-immobilized complex is formed between the third component and the small molecule analyte of the fluid sample by specific binding between the detection molecule and the small molecule analyte of the fluid sample; separating the immobilized complex from the non-immobilized complex; detecting a signal from the labeled immobilized complex, the labeled non-immobilized complex, or both the labeled immobilized complex and the labeled non-immobilized complex; and comparing the signal to a standard, thereby quantitating the detected small molecule analyte in the sample.

Methods of detecting a small molecule analyte in a sample are provided according to aspects of the present invention which include providing a first component that includes a receptor, wherein the first component is immobilized on a solid phase support, and the receptor does not specifically bind to the small molecule analyte; providing a second component that includes a ligand for said receptor, said ligand conjugated to a conjugated small molecule analyte; providing a third component including a detection molecule conjugated to a label, the detection molecule capable of specific binding with the small molecule analyte and does not specifically bind to the first component; contacting the second component and the immobilized first component under specific binding reaction conditions, whereby the second component is immobilized on the solid phase support by specific binding of the receptor and the ligand, producing an immobilized second component; contacting a fluid sample containing or suspected of containing the small molecule analyte and the immobilized second component with the third component under specific binding reaction conditions, whereby an immobilized complex is formed between the third component and the immobilized second component by the binding between the detection molecule and the conjugated small molecule analyte, and whereby a non-immobilized complex is formed between the third component and the small molecule analyte of the fluid sample by specific binding between the detection molecule and the small molecule analyte of the fluid sample; separating the immobilized complex from the non-immobilized complex; and detecting a signal from the label of the immobilized complex or from the non-immobilized complex, thereby detecting the small molecule analyte in the sample. Optionally, the signal is compared to a standard, thereby quantitating the small molecule analyte in the sample

Methods of detecting and/or quantitating a small molecule analyte in a sample are provided according to aspects of the present invention in which the small molecule analyte is selected from the group consisting of: 8-hydroxyguanosine (8-HOdG), 8-hydroxyguanosine, 8-hydroxy-2 and two or more thereof.

Methods of detecting and/or quantitating a small molecule analyte in a sample are provided according to aspects of the present invention in which the receptor includes an antibody, the antigen-binding portion of an antibody, a lectin, fibronectin, protein A, protein G, avidin, steptavidin, or two or more thereof.

Methods of detecting and/or quantitating a small molecule analyte in a sample are provided according to aspects of the present invention in which the ligand includes an antibody, the antigen-binding portion of an antibody, a protein, a sugar, biotin, a biotin labeled protein, or two or more thereof.

Methods of detecting and/or quantitating a small molecule analyte in a sample are provided according to aspects of the present invention in which the fluid sample is saliva, urine, plasma, serum, hair, feathers, fecal, samples, CSF, milk, isolated DNA, isolated RNA, a cell lysate, a tissue sample lysate, cell culture medium or two or more thereof.

Methods of detecting and/or quantitating a small molecule analyte in a sample are provided according to aspects of the present invention in which the detection molecule includes either an antibody specific for the small molecule analyte or an antigen-binding portion of the foregoing antibody.

Methods of detecting and/or quantitating a small molecule analyte in a sample are provided according to aspects of the present invention in which the label is an enzyme, a fluorescent moiety, a dye, a chemiluminescent moiety, a magnetic particle, a radioisotope, a chromophore or combinations of any two or more thereof.

Methods of detecting and/or quantitating a small molecule analyte in a sample are provided according to aspects of the present invention in which the solid phase support includes a polystyrene, support.

Methods of detecting and/or quantitating a small molecule analyte in a sample are provided according to aspects of the present invention in which contacting a fluid sample containing or suspected of containing the small molecule analyte with the immobilized second component is performed concurrently with contacting a fluid sample containing or suspected of containing the small molecule analyte with the third component.

Kits for the detection and/or quantitation of a small molecule analyte are provided according to aspects of the present invention which include a first component includes a receptor; a second component that includes a ligand for said receptor, said ligand conjugated to the small molecule analyte; and a third component that includes a detection molecule conjugated to a label, the detection molecule capable of specific binding with the small molecule analyte.

Kits for the detection and/or quantitation of a small molecule analyte are provided according to aspects of the present invention in which the first component is immobilized on a solid phase.

Kits for the detection and/or quantitation of a small molecule analyte are provided according to aspects of the present invention in which the solid phase is a polystyrene support.

Kits for the detection and/or quantitation of a small molecule analyte are provided according to aspects of the present invention in which the second component is immobilized on the solid phase by the binding between the immobilized receptor and the ligand.

Kits for the detection and/or quantitation of a small molecule analyte are provided according to aspects of the present invention in which the small molecule analyte is 8-hydroxyguanosine, 8-hydroxyguanosine, 8-hydroxy-2′-deoxyguanosine, or two or;more thereof.

Kits for the detection and/or quantitation of a small molecule analyte are provided according to aspects of the present invention in which the receptor includes an antibody, an antigen-binding portion of an antibody, a lectin, fibronectin, protein A, protein G, avidin, steptavidin, or two or more thereof.

Kits for the detection and/or quantitation of a small molecule analyte are provided according to aspects of the present invention in which the ligand includes an antibody, an antigen-binding portion of an antibody, a sugar, gelatin, biotin, a biotin labeled protein, or two or more thereof.

Kits for the detection and/or quantitation of a small molecule analyte are provided according to aspects of the present invention in which the small molecule analyte is 8-hydroxyguanosine, 8-hydroxyguanosine, 8-hydroxy-2′-deoxyguanosine, or two or more thereof.

Kits for the detection and/or quantitation of a small molecule analyte are provided according to aspects of the present invention in which the detection molecule is an antibody specific for 8-hydroxyguanosine, 8-hydroxyguanosine, 8-hydroxy-2′-deoxyguanosine, or two or more thereof, or the detection molecule includes the antigen-binding portion of such an antibody.

Kits for the detection and/or quantitation of a small molecule analyte are provided according to aspects of the present invention in which the label is an enzyme, a dye, a fluorescent moiety, a chemiluminescent moiety, a magnetic particle, a radioisotope, a chromophore or combinations of any two or more thereof.

### BRIEF DESCRIPTION OF THE DRAWINGS

FIG. 1 is a graph showing UV/visible spectrum slightly modified compared to a UV/visible spectrum of BSA alone due to the conjugate of oxidized 8-hydroxyguanosine with BSA;

FIG. 2 is a graph showing the change of bound peroxidase signal with 1000 ng/in 750 ng/mL or 500 ng/mL, of mouse primary antibody specific for 8-hydroxyguanosine;

FIG. 3 is a graph showing lack of reproducibility of an assay to measure a small molecule analyte (8-hydroxyguanosine) using a microtiter plate coated with varying concentrations of the analyte conjugate (8-hydroxyguanosine:BSA conjugate)

FIG. 4 is a graph showing the spectra of the 8-hydroxyguanosine:rabbit IgG conjugate;

FIG. 5 is a graph showing results using an assay according to aspects of the present invention;

FIG. 6 is a graph showing results of format variations using identical reagents and plates according to aspects of the present invention;

FIG. 7 is a graph showing reproducibility of an assay to measure a small molecule analyte according to aspects of the present invention;

FIG. 8 is a diagram depicting steps A, B, C, D, E, and F according to aspects of the present invention. The diagram depicts A; a first component including a receptor, wherein the first component is immobilized on a solid phase support and contacting a fluid sample containing a small molecule analyte; B contacting the second component and the immobilized first component under specific binding reaction conditions, whereby the second component is immobilized on the solid phase support by specific binding of the receptor and the ligand, producing an immobilized second component; C; contacting a fluid sample containing or suspected of containing the small molecule analyte and the immobilized second component with the third component under specific binding reaction conditions, whereby a labeled immobilized complex is formed between the third component and the immobilized second component by the binding between the detection molecule and the conjugated small molecule analyte, and whereby a labeled non-immobilized complex is formed between the third component and the small molecule analyte of the fluid sample by specific binding between the detection molecule and the small molecule analyte of the fluid sample; D; separating the immobilized complex from the non-immobilized complex, thereby resulting in a container, such as a well of a microtiter plate, containing only immobilized complex as depicted in E and a container containing only non-immobilized complex as depicted in F. The diagram further depicts the detection of a signal from the label of the immobilized complex in E and/or the non-immobilized complex in F; and

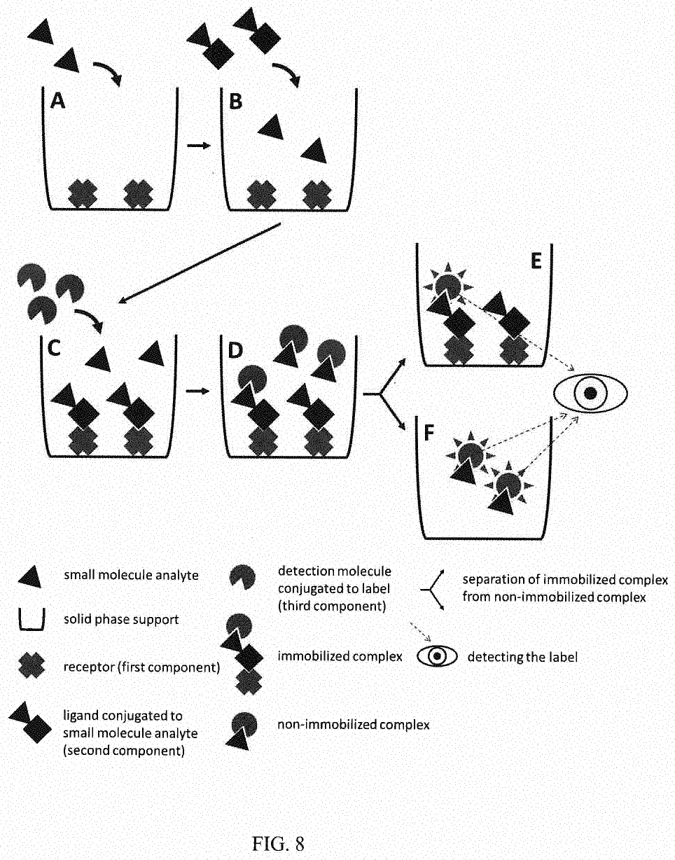

FIG. 9 shows linearity of the assay.

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W, Russell, Molecular Cloning; A Laboratory Manual, Cold Spring Harbor Laboratory Press: 3rd Ed., 2001; F. M, Ausubel, Ed., Short Protocols in Molecular Biology, Current Protocols; 5th Ed., 2002; B. Alberts et al., Molecular Biology of the Cell, 4th Ed., Garland, 2002; and D. L. Nelson and M. M. Cox, Lehninger Principles of Biochemistry, 4th Ed., W. H. Freeman & Company, 2004.

The articles “a” and “an” are used herein to refer to one or to more than one (i.e., to at least one) of the grammatical object of the article. By way of example, “an element” means one element or more than one element.

Methods and kits according to aspects of the present invention provide improved detection and quantification of small molecule analytes.

Methods and kits according to particular aspects of the present invention provide improved detection and quantification of guanosine derivatives in a biological sample, wherein the guanosine derivatives are produced by oxidation of DNA.

Methods for detecting a small molecule analyte in a sample according to aspects of the present invention include: providing an immobilized first component comprising a receptor, wherein the first component is immobilized on a solid phase support; providing a second component comprising a ligand for the receptor, the ligand conjugated to the small molecule analyte; providing a third component including a detection molecule conjugated to a label, the detection molecule capable of specific binding with the small molecule analyte to be detected in the assay; contacting the second component and the immobilized first component under specific binding reaction conditions for binding of the receptor of the first component and the ligand of the second component, whereby the second component is immobilized on the solid phase support by specific binding of the receptor and the ligand, producing an immobilized second component; contacting a fluid sample containing or suspected of containing the small molecule analyte with the solid phase support; contacting the third component with the immobilized second component and the fluid sample under specific binding reaction conditions for binding the small molecule analyte (in the fluid sample and in the second component conjugate) and the detection molecule, whereby an immobilized complex is formed between the third component and the immobilized second component by the specific binding between the detection molecule and the small molecule analyte of the second component, and whereby a non-immobilized complex is formed between the third component and the small molecule analyte by specific binding between the detection molecule and the small molecule analyte in the fluid sample; separating the immobilized complex from the non-immobilized complex; and detecting the label of the immobilized complex and/or the non-immobilized complex, thereby detecting the small molecule analyte in the sample.

The term “small molecule analyte” as used herein refers to organic molecules characterized by a molecular weight below about 900 Da, such as in the range of 10 g/mol-900 g/mol. Small molecule analytes to be assayed can be any small molecule of interest, including naturally occurring substances, non-natural synthesized substances and metabolites.

Specific examples of small molecule analytes detected according to aspects of inventive methods are small peptides examples of which are: oxidized or reduced glutathione, steroids exemplified by but not limited to estradiol, progesterone, cortisol, andrenostenedione, dehydroepiandrosterone, testosterone, diethylstilbestrol, dexamethasone, nandrolone, stanozolol, methandienone, boldenone; vitamins, hormones exemplified by but not limited to hydroxycholecalciferol, thyroxine, tri-iodothyronine, carnitine, acylcarnitine, chloroquine; cholesterol; amino acids and metabolites exemplified by, but not limited to, histidine, urocanic acid, homocysteine, phenylalanine, tyrosine and tryptophan; drug molecules exemplified by but not limited to heparin, biopterin; clenbuterol, mefloquine, theophylline, tetrahydrocannabinol, amphetamine, methamphetamine, phencyclidine, barbiturates; drug of abuse and metabolites thereof exemplified by, but not limited to, heroin, codeine, morphine, opium, meperidine, cocaine, nicotine, ethanol; signaling molecules exemplified by hut not limited to cyclic nucleotides, prostaglandins, leukotrienes, lipids, and fatty acids.

Specific examples of small molecule analytes detected according to aspects of inventive methods are guanine derivatives representing oxidative DNA damage.

Such guanine derivatives include 8-hydroxyguanosine, 8-hydroxyguanosine and 8-hydroxy-2′-deoxyguanosine.

According to particular aspects, cortisol is a small molecule analyte detected according to aspects of inventive methods.

The term “immobilized” as used herein refers to binding of first, first and second or first, second and third components so that they are retained in contact with a solid phase support when washed with phosphate buffered saline for 30 seconds at 25° C.

The term “receptor” as used herein is any compound or composition capable of recognizing and binding to a particular spatial and polar organization of a molecule. Illustrative receptors include antibodies, enzymes, poly(nucleic acids), complement component, thyroxine binding globulin, lectins, fibronectin, nucleic acids, protein A, protein G, avidin, streptavidin, and the like.

The term “antibody” includes whole immunoglobulin molecules having a single specificity as is conventional in the art. An antibody can be IgA, IgD, IgE, IgG, IgM, subclasses thereof, and can be derived from various sources, including rat, mice, goat, human, etc. In addition, the term is intended to include chemically prepared fragments, such as Fab, F(ab)′, and/or F(ab)2 fragments, of such molecules and recombinantly prepared equivalents thereof, such as “single chain antibody fragments” or ScFv fragments. Each type of antibody described herein can be monoclonal or polyclonal. Monoclonal antibodies include those molecules generally prepared using conventional hybridoma technology, but they can also be prepared by electrofusion, viral transformation and other procedures known in the art. In certain aspects, the receptor is a generic or secondary antibody, such as a goat, rat, sheep, donkey or mouse antibody to rabbit IgG, a rabbit, goat, rat, donkey or mouse antibody to sheep IgG, a rabbit, goat, rat, sheep or donkey antibody to mouse IgG, or the like. In certain aspects, the receptor is an anti-rabbit IgG antibody.

Antibodies and methods for preparation of antibodies are well-known in the art. Details of methods of antibody generation and screening of generated antibodies for substantially specific binding to an antigen are described in standard references such as E. Harlow and D, Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; and B. K. C. Lo, Antibody Engineering: Methods and Protocols, Methods in Molecular Biology, Humana Press, 2003.

According to aspects of the invention, a method for detecting a small molecule analyte in a sample includes providing a second component, the second component including a ligand for a corresponding, receptor included in the first component.

The term “ligand” as used herein is any organic molecule for which a receptor naturally exists or can be prepared. Ligands include, but are not limited to, polypeptides, proteins including enzymes, antibodies, antigenic proteins, glycoproteins, lipoproteins, streptavidin, avidin, fluorescein, digoxygenin, dinitrophenol, components of cells and viruses, nucleic acids, such as single- and double-stranded oligonucleotides, lectins, carbohydrates (such as polysaccharides), biotin, and other materials readily apparent to one skilled in the art. In certain aspects, the ligand is an antibody, including anti-antibodies.

In certain aspects, the ligand is protein A, protein G, avidin, streptavidin, biotin, rabbit IgG, donkey lgG, goat IgG, sheep IgG, guinea pig IgG, chicken IgY, rat IgG, horse IgG, pig IgG, bovine IgG, human IgG, non-human primate IgG or mouse IgG. For example, in some aspects of the invention, the ligand is a rabbit antibody.

Examples of ligand/receptor complexes, that is, a specific binding reaction product between a ligand and corresponding receptor, include, but are not limited to, antibody/antigen, antibody/hapten, antibody/antibody, avidin/biotin, streptavidin/biotin, sugar/lectin, gelatin/fibronectin, nucleic acid/complementary nucleic, acid and IgG/Protein A or IgG/Protein G complexes.

Further examples of ligand/receptor complexes include fluorescein/an anti-fluorescein antibody, digoxygenin/an anti-digoxygenin antibody, dinitrophenol/an anti-dinitrophenol antibody.

According to aspects where the receptor is Protein A or Protein G, an inventive assay includes use of a third component which includes a detection molecule conjugated to a label, the detection molecule capable of specific binding with the small molecule analyte, wherein the detection molecule is not capable of specific binding with the Protein A or Protein G. Notably, according to aspects where the receptor is Protein A or Protein G, an inventive assay;includes use of a third component which includes a detection molecule conjugated to a label, the detection molecule capable of specific binding with the small molecule analyte, wherein the detection molecule is not an intact antibody. The detection molecule in this aspect is illustratively an antibody fragment which is not capable of specific binding to Protein A or Protein G. An antibody fragment which is not capable of specific binding to Protein A or Protein G can be an antibody fragment which retains an antigen binding site but not an Fe domain, such as an Fab fragment of an antibody.

In certain aspects, ligand/receptor complexes include a goat anti-rabbit IgG/rabbit rat anti-rabbit IgG/rabbit IgG, donkey anti-rabbit IgG/rabbit IgG or mouse anti-rabbit IgG/rabbit IgG, donkey anti-sheep IgG/sheep IgG, goat anti-sheep IgG/sheep IgG, rabbit anti-sheep IgG/sheep IgG, mouse anti-sheep IgG/sheep IgG, rat anti-sheep IgG/sleep IgG, goat anti-mouse IgG/mouse IgG, donkey anti-mouse IgG/mouse IgG, sheep anti-mouse IgG/mouse IgG, rabbit anti-mouse IgG/mouse IgG, rat anti-mouse IgG/mouse IgG or the like.

The term “specific binding,” when used in relation to a receptor and ligand, refers to hybridization off particular receptor to a ligand without substantial binding to other molecules in a sample. Specific binding of a receptor and ligand can be characterized by a dissociation constant indicative of affinity between the receptor and ligand. Dissociation constants indicative of specific binding between a receptor and ligand are generally in the range of about 10⁻⁴ M to about 10⁻¹² or less, and preferably in the range of about 10⁻⁸ M to about 10⁻¹² M or less.

In some aspects of the method for detecting a small molecule analyte in a sample, two components are conjugated to each other. According to particular aspect, the ligand is conjugated to the small molecule analyte. According to farther aspects, the detection molecule is conjugated to a label, “Conjugation” is any process wherein two subunits are linked together to form a conjugate. For example, a covalent bond is a form of conjugation.

Methods for forming conjugates are well-known in the art. In a non-limiting example, 1-Ethyl-3-[3-dimethylaminopropyl]carbodiimide hydrochloride, EDC or EDAC chemistry, can be used to form conjugates

In the context of methods and compositions described herein, one type of “conjugate” is a molecule including a ligand and a small molecule analyte bound together, optionally through a linker, to form a single structure.

According to aspects of the present invention, the ligand is directly bound to the small molecule analyte. For example, the ligand can be an antibody, antigen, hapten, avidin, biotin, streptavidin, sugar, lectin, gelatin, fibronectin, nucleic acid. Protein A or Protein G conjugated directly to the small molecule analyte.

In a specific example, a hydroxylated derivative of guanosine, 8-hydroxyguanosine (8HOG) is oxidized using sodium periodate to form aldehydes. The aldehydes react with amine groups on proteins and form a stable bond, thereby forming a ligand/small molecule analyte conjugate used as a second component in assays disclosed herein.

The conjugate can be made either by a direct connection, such as a chemical bond, between the ligand and the small molecule analyte, or an indirect connection, such as by use of a linker. The linker is not limited in type and can have, for example, a linear or branched carbon backbone of CI-C20. Further examples of linkers are oligo- or polysaccharide linkers and oligo- or polynucleotide linkers. An included linker can vary, depending upon the particular ligand and small molecule analyte and one skilled in the art will recognize an appropriate linker without undue experimentation.

In a particular aspect according to the present invention, the linker is a protein, such as ara immunoglobulin, BSA or ovalbumin conjugated to a ligand, such as an antibody, antigen, hapten, avidin, biotin, streptavidin, sugar, lectin, gelatin, fibronectin, nucleic acid, Protein A or Protein G and more particularly such as avidin, biotin, streptavidin or a nucleic acid.

In one aspect of the present invention, 8-hydroxyguanosine, 8-hydroxyguanosine, 8-hydroxy-T-deoxyguanosine, or any two or more thereof, is directly conjugated to the ligand. In another aspect of the invention, 8-hydroxyguanosine, 8-hydroxyguanosine, 8-hydroxy-2′-deoxyguanosine, or any two or more thereof, is indirectly conjugated to the ligand.

According to aspects of the method for detecting, a small molecule analyte, 8-hydroxyguanosine, 8-hydroxyguanosine, 8-hydroxy-2′-deoxyguanosine, or any two or more thereof, is directly or indirectly bound to an antibody or other ligand capable of specific binding to a receptor immobilized on the solid phase support.

As noted above, a third component used in a method for detecting a small molecule analyte includes a detection molecule conjugated to a label. The detection molecule is capable of specific binding with a small molecule analyte. In some aspects, the detection molecule is an antibody specific for a guanosine derivative. In certain aspect, the detection molecule is an antibody specific for 8-hydroxyguanosine, 8-hydroxyguanosine, 8-hydroxy-2′-deoxyguanosine, or combinations thereof.

A “label” conjugated to a detection molecule produces or can, be induced to produce a detectable signal.

Examples of labels illustratively include a fluorescent moiety, a chemiluminescent moiety, a bioluminescent moiety, a dye, a magnetic particle, an enzyme, a radioisotope and a chromophore. A reagent combined with a label is a “labeled reagent.”

The label may be isotopic or nonisotopic. By way of example and not limitation, the label can be a part of a catalytic reaction system such as enzymes, enzyme fragments, enzyme substrates, enzyme inhibitors, coenzymes, or catalysts; part of a chromogen system such as fluorophores, dyes, chemilumincseers, luminescers, or sensitizers; a dispersible particle that can be, non-magnetic or magnetic, a solid support, a liposome, a ligand, a receptor, a hapten radioactive isotope, and so forth. In certain embodiments, the label is an enzyme, a fluorescent probe, a chemiluminsecent probe, a metal, a non-metal colloidal particle, a polymeric dye particle, or a pigment particle.

Enzymes, enzyme fragments, enzyme inhibitors, enzyme substrates, and other components of enzyme reaction systems can be used as labels. Where any of these components is used as a label, a chemical reaction involving one of the components is part of the signal producing system. When enzymes are employed, molecular weights of the label typically range from about 10,000 to 600,000, more usually from about 10,000 to 300,000, and the involved reactions will be for the most part, hydrolysis or redox reactions.

Coupled catalysts can also involve an enzyme with a non-enzymatic catalyst. The enzyme can produce a reactant, which undergoes a reaction catalyzed by the non-enzymatic catalyst or the non-enzymatic catalyst may produce a substrate (includes coenzymes) for the enzyme. A wide variety of non-enzymatic catalysts as are known in the art may be employed. The enzyme or coenzyme employed provides the desired amplification by producing a product, for example, which absorbs light, e,g., a dye, or emits light upon irradiation, e.g., a fluorescer. Alternatively, the catalytic reaction can lead to direct light emission, e.g., chemiluminescence. A large number of enzymes and coenzymes for providing such products are known in the art.

Enzymes useful are labels include hydrolases, transferases, lyases, isomerases, ligases or synthetases and oxidoreductases, preferably hydrolases. Optionally, a luciferase may be used, such as firefly luciferase or bacterial luciferase. Primarily, the enzymes of choice, based on the I.U.B. classification are: Class 1, oxidoreductases and Class 3, Hydrolases; particularly in class 1, the enzymes of interest are dehydrogenases of Class 1.1, more particularly 1.1.1, 1.1.3, and 1.1.99 and peroxidases, in Class 1.11. Of the hydrolases, particularly Class 3.1, more particularly 3,1.3 and Class 3.2, more particularly 3.2.1 are useful. Optionally, malate dehydrogenase, glucose-6-phosphate dehydrogenase, or lactate dehydrogenase is used. According to particular aspects, glucose oxidase is used. According to further aspects, horse radish peroxidase is used. According to still further aspects, alkaline phosphatase, beta-glucosidase or lysozyme is used.

The label can also be fluorescent either directly or by virtue of fluorescent compounds or fluorescers bound to a particle or other molecule in conventional ways. The fluorescent labels wall be bound to, or functionalized to render them capable of binding (being conjugated) to, optionally through a linking group, cyclosporin or antibodies or receptors for cyclosporin. The fluorescers of interest will generally emit light at a wavelength above about 350 nm, usually above about 400 nm and preferably above about 450 nm. Desirably, the fluorescers have a high quantum efficiency, a large Stokes shift, and are chemically stable under the conditions of their conjugation and use. The term luminescent label is intended to include substances that emit light upon activation by electromagnetic radiation, electro chemical excitation, or chemical activation and includes fluorescent and phosphorescent substances, scintillators, and chemilurninescent substances.

Fluorescers of interest fall into a variety of categories having certain primary functionalities. These primary functionalities include 1- and 2-aminonaphthalene, p,p-diaminostilbenes, pyrenes, quaternary phenanthridine salts, 9-aminoaeridines, p,p′-diaminostilbenes imines, anthracenes, oxacarboxyanme, inerocyanine, 3-aminoequil perylene, bis-benzoxazole, bis-p-oxazolyl benzene, 1,2-benzophenazine, retinol, bis-3-aminopyridinium salts, hellebrigenin, tetracycline, sterophenol, benzimidazolylphenylamine, 2-oxo-3-chromen, indole, xanthene, 7-hydroxycoumarin, 4,5-benzimidazoles, phenoxazine, salicylate, strophanthidin, porphyrins, triarylmethanes, flavin and rare earth chelates, oxides, and salts.

Energy absorbers or quenchers can be employed either separately or in conjunction with one another. The absorber or quencher can additionally be bound to a solid insoluble particle of at least about 50 mn in diameter. When the distance between the absorber and the quencher resulting from specific binding events (such as antibody-antigen binding) too small, the fluorescence of the absorber is quenched by the quencher. The quencher may be the same or different, usually different, from the fluorescer.

An alternative source of light as a detectable signal is a chemiluminescent source, and, therefore, a label can be a chemiluminescent compound, The chemiluminescent source involves a compound, which becomes electronically excited by a chemical reaction and may then emit light which serves as the detectable signal or donates energy to a fluorescent acceptor.

A diverse number of families of compounds have been found to provide chemiluminescence under a variety of conditions. One family of compounds is 2,3-dihydro-1,4-phthalazinedione. The most popular compound is lurninol, which is the 5-amino analog of the above compound. Other members of the family include the 5-amino-6,7,8-trimethoxy- and the dimethylainine-[ca]benzo analog. These compounds can be made to luminesce with alkaline hydrogen peroxide or calcium hypochlorite and base. Another family of compounds is the 2,4,5-triphenylimidazoles, with lophine as the common name for the parent product. Chemiluminescent analogs include para-dimethylamino- and para-methoxy-substituents. Chemiluminescence may also be obtained with geridinium esters, dioxetanes, and oxalates, usually oxalyl active esters, e.g., p-nitrophenyl and a peroxide, e.g., hydrogen peroxide, under basic conditions. Alternatively, luciferins may be used in conjunction with luciferase or lucigenins.

Any detection method or system operable to detect a label can be used in methods according to embodiments of the present invention and such appropriate detection methods and systems are well-known in the art, illustratively including spectroscopic, optical, photochemical, biochemical, enzymatic, electrical and/or immunochemical detection methods and systems.

The term “solid-phase support” refers to a porous or non-porous member which is substantially non-soluble in an aqueous medium included in an assay mixture. A solid-phase support can be solid, semi-solid, gel or a mixture thereof.

A solid phase support on which a first component is immobilized can be in any of various forms or shapes, including planar, such as chips and plates; and three-dimensional, such as particles, beads, microtiter plates, wells, microtiter wells, pins, mesh, fibers, a membrane, such as a nitrocellulose membrane; and a container and the like.

The solid phase support can be any of various materials such as glass; plastic, such as polypropylene, polystyrene, nylon; paper; metal; silicon; nitrocellulose; agarose; dextran; polyacrylamide; or any other material to which a first component can be attached for use in an assay.

The solid phase support can be hydrophilic or capable of being rendered hydrophilic and can be any of various materials including natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly(vinyl chloride), polyacrylamide, cross linked dextran, agarose, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials; glass, ceramics, metals, inorganic powders such as silica, mapesium sulfate, and alumina; and the like.

In certain aspects, the solid phase support is a polystyrene support or includes a polystyrene support

The surface of the solid phase support, is capable of binding the first component through specific or non-specific covalent or non-covalent interactions

The surface of the solid phase support includes reactive functional groups, or is capable of being polyfunctionalized to include reactive functional groups, capable of reacting with the first component to immobilize the first component on the solid phase support.

Reactive functional groups include, but are not limited to, carboxy, 2-substituted ethylsulfonyl, vinylsulfonyl, epoxy, aldehyde, active halo atoms, amino, hydrazine and active esters such as succinimidoxycarbonyl, carboxyl, amine, carboxylate, halide, ester, alcohol, carbamide, chloromethyl, sulfur oxide, nitrogen oxide, and/or tosyl functional groups.

Attachment of the first component to the solid phase support can be accomplished using any of a variety of conventional procedures, such as coating, to adsorb the first component by non-specific binding or reacting the first component with reactive functional groups on the support as is well known in the art.

Alternatively, the first component can be attached to the solid phase support via reaction with linking groups attached thereto, and such linking groups can be chemical moieties extending from the support to which the first component can be conjugated.

In some aspects, the solid phase support is coated with a large excess of the first component such that all specific and non-specific binding sites on the solid phase support are covalently or non-covalently bound to the first component.

In certain aspects, the solid phase support is coated with an amount of the first component sufficient to saturate substantially all non-specific binding sites on the solid phase support. The term “substantially all non-specific binding sites on the solid phase support” means that fewer than 0.01% of the non-specific binding sites on the solid phase support remain unbound to the first component. An amount of the first component sufficient to saturate substantially all nonspecific binding sites on the solid phase support is typically in the range of 1-100 μg/mL of the first component, optionally in the range of 5-50 μg/mL of the first component and further optionally in the range of 10-20 μg/mL of the first component.

In some aspects of the method for detecting a target small molecule analyte in a sample, the method includes contacting the second component with the first component immobilized on the solid phase support. The second component is immobilized on the solid phase by the binding between the immobilized receptor and the ligand. As described above, suitable receptor/ligand complexes include, but are not limited to, antibody/antigen, antibody/hapten, antibody/antibody, avidin/biotin, streptavidin/biotin, sugar/lectin, gelatin/fibronectin, nucleic acid/complementary nucleic acid and IgG/protein, A or IgG/Protein G complexes.

In some aspects of the method for detecting a target small molecule analyte in a fluid sample, the method includes contacting a fluid sample suspected of containing a target guanosine derivative with the solid phase support having the first and/or second components immobilized thereon.

A fluid sample is typically a fluid or tissue of a mammalian subject, including a primate or human subject. The term “subject” as used herein refers to any animal subject, such as humans, non-human primates, cats, dogs, sheep, cows, goats, horses, pigs, poultry, binds, rabbits and rodents. Subjects can be either gender and can be any age.

The small molecule analyte to be detected may be present in any of, a wide variety of fluid samples, or aqueous solutions, of animal or human body fluids, tissues or waste products including, but not limited to, whole blood, serum, plasma, lymph fluid, bile, urine, spinal fluid, lacrimal fluid, interstitial fluid, swab specimens, stool specimens, semen, vaginal secretions, saliva, crevicular fluid, tissue culture media samples, hair, feathers, fecal samples, CSF, milk, and other sample types readily apparent to one skilled in the art.

A fluid sample containing or suspected of containing DNA damage oxidation products to be detected in an inventive method may be any of a wide variety of fluid samples, or aqueous solutions, of animal or human body fluids, tissues or waste products including, but not, limited to, blood, saliva, urine, plasma, serum, isolated DNA, isolated RNA, a cell lysate, a tissue sample lysate, cell culture medium, hair, feathers, fecal samples, CSF, milk, or two or more thereof.

A sample for use, in methods of the present invention to detect DNA damage oxidation products can also be DNA and/or RNA isolated from an in vitro cell or subject sample.

The term “isolated” refers to DNA and/or RNA at least partly separated from substances with which the DNA and/or RNA naturally occur, such as cellular proteins, lipids and/or carbohydrates. The term “isolated” does not implicate absolute purity of the DNA and/or RNA. In embodiments, the isolated DNA and/or RNA in a sample represent at least 50%, 60%, 70%, 80%, 90%, 95%, 99% or greater of the total organic content of the sample.

The size of the fluid sample can vary widely and may be selected or adjusted according to the particular assay conditions.

In some aspects of the method for detecting a target small molecule analyte in a sample, the solid phase support having the first and second components immobilized thereon is contacted the third component and the fluid sample in an aqueous medium, wherein the fluid sample contains, or is suspected of containing, the target small molecule analyte. The target small molecule analyte in the fluid sample and the small molecule analyte conjugated to the ligand compete for available binding sites on the detection molecule of the third component, thereby forming specific binding complexes: 1) an immobilized complex is formed between the third component and the immobilized second component by the binding between the detection molecule and the small molecule analyte of the second component; and 2) a non-immobilized complex is formed in the aqueous medium between the third component and the target small molecule analyte by the binding between the detection molecule and the target small molecule analyte.

In certain aspects of the method, the third component is contacted with the second component at a stoichiometry of either 1:1 or less than 1:1, wherein the stoichiometry refers to the number of detection molecules in the third component relative to the number of small molecule analytes in the second component that are capable of binding detection molecules of the third component. In general, each molecule of the third component contains a single detection molecule, for example, to prevent crosslinking between the second component and small molecule analyte of the sample. Each molecule of the second component contains 1 or more than 1 conjugated small molecule analyte. Depending on the conjugation strategy, however, less than each conjugated small molecule analyte may be accessible to bind a detection molecule. The precise stoichiometry is not particularly limiting so long as the number of molecules of the first component, second component, and third component used to detect the small molecule analyte in a specific sample is approximately equal to the number of molecules of the first component, second component, and third component used to detect the small molecule analyte in relevant controls.

In certain aspects of the method for detecting a target small molecule analyte in a sample, the fluid sample and the third component are present together in an aqueous medium which is contacted with the solid phase support having the first and second component immobilized thereon. In other aspects of the method for detecting a target small molecule analyte in a sample, the aqueous medium is first contacted with the solid phase support after which the fluid sample and the third component are added, together or sequentially.

In further aspects of the method for detecting a target small molecule analyte in a sample, the aqueous medium contains either the fluid sample or the third component and this mixture is contacted with the solid phase support, after which the remaining reaction element, either the fluid sample or the third component is added.

According to aspects of the method for detecting a target small molecule analyte in a sample, the immobilized complex attached to the solid phase support is separated from the non-immobilized complex present in the aqueous medium. The immobilized complex attached to the solid phase support formed between the third component and the immobilized second component by the binding between the detection molecule and the conjugated small molecule analyte of the second component is then separated from any non-immobilized complex that formed between third component and the target small molecule analyte by the binding between the labeled detection molecule and the target small molecule analyte.

Separation of immobilized complexes from non-immobilized complexes can be accomplished using any suitable equipment and procedure.

For example, the aqueous medium can he removed from contact with the solid phase support and the solid phase support may be rinsed one or more times to further remove any remaining non-immobilized complexes.

Liquid containing the non-immobilized complexes may be aspirated or poured from the solid phase support thereby separating the immobilized complexes from the non-immobilized complexes.

Optionally, where the solid support is particles of a suitable material, such as polymeric particles as described above, separation of immobilized complexes from non-immobilized complexes is achieved by filtration or centrifugation to separate the solid phase support and immobilized complexes from the aqueous medium and non-immobilized complexes. The immobilized complexes and/or non-immobilized complexes are optionally placed in or retained in a container, such as a well of a microtiter plate or a microtube.

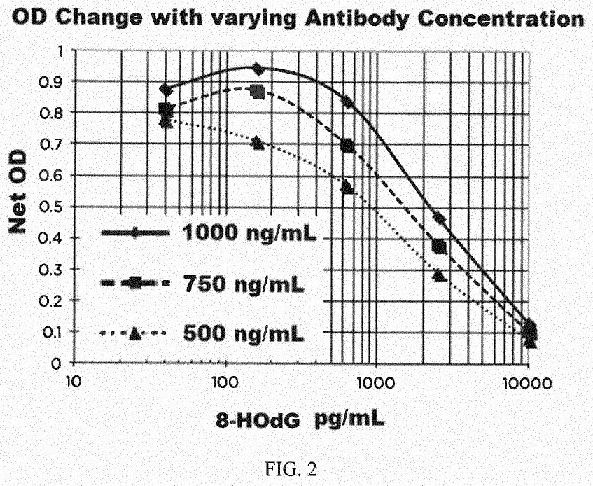

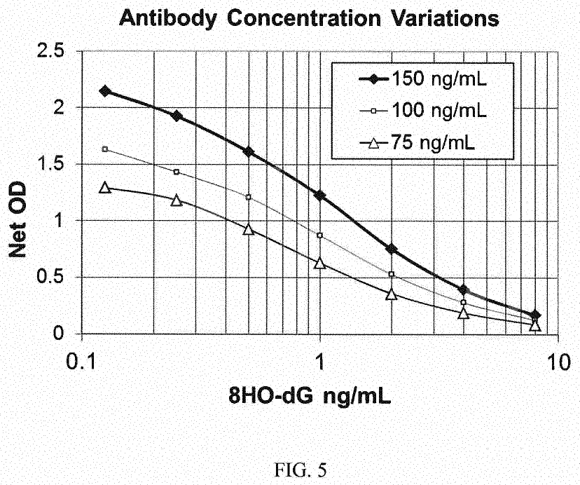

According to aspects of the method for detecting a target small molecule analyte in a sample, the method includes detecting the label from the immobilized complex, the non-immobilized complex, or a combination thereof. Signal is generated from the presence of the label in the immobilized complex in an indirect proportion to the amount of target small molecule analyte in the fluid sample. The target small molecule analyte in the fluid sample competes with the small molecule analyte conjugated to the ligand of the second component for binding with the labeled detection molecule of the third component, and the amount of signal is quantified by an appropriate method depending on the label. Measurement of signal from the label in the immobilized complex results in an inverse correlation between the amount of target small molecule analyte in the sample and the generated signal. FIGS. 2 and 5 depict typical standard curves. Measurement of signal from the label in the non-immobilized complex will yield a positive correlation between the amount of target small molecule analyte in the sample and the generated

To determine the appropriate amounts of each reactant, e.g., first, second and third components, to be used in an assay according to aspects of the present invention, one or more dilution curves can be obtained for each binding pair, e.g., antibody/antigen, antibody/hapten, antibody/antibody, streptavidin/biotin, sugar/lectin, gelatin/fibronectin, nucleic acid/complementary nucleic acid and IgG/Protein A or IgG/Protein G. A dilution curve is generated by reacting a fixed amount of ligand with various amounts of receptor, for example, serial doubling dilutions of the receptor. For the dilution curve, the ligand can be labeled so that the amount of bound ligand is differentiated from the amount of free ligand. Generally, the amount of receptor sufficient to bind approximately 50% of the fixed amount of ligand is the approximate amount of receptor used to generate a standard curve.

A standard curve is generated according to aspects of the present invention. A standard curve is determined by reaction of a fixed amount of a labeled ligand and receptor with varying amounts of unlabeled ligand.

Assays according to aspects of the present invention are generally carried out under conditions suitable for biomolecular interaction and activity. The assay mixture generally includes an aqueous medium. The pH of the assay mixture is usually in the range of about pH 4-pH 11, preferably in the range of about pH 5-pH 10, and more preferably in the range of about pH 6-pH 8. One or more buffers can be included in the assay mixture illustratively including, but not limited to, borate, cacodylate, carbonate, citrate, HEPES, MES, MOPS, phosphate, phosphate buffered saline, PIPES, TAPS, TES and Tris buffers. The reagents included in a reaction are reacted at suitable temperature, generally in the range of from about 4° to about 35° C., and preferably at room temperature. The reaction time can vary from a few seconds to several days, typically in the range of one hour—overnight. For example, the reaction time may be about 2 hours.

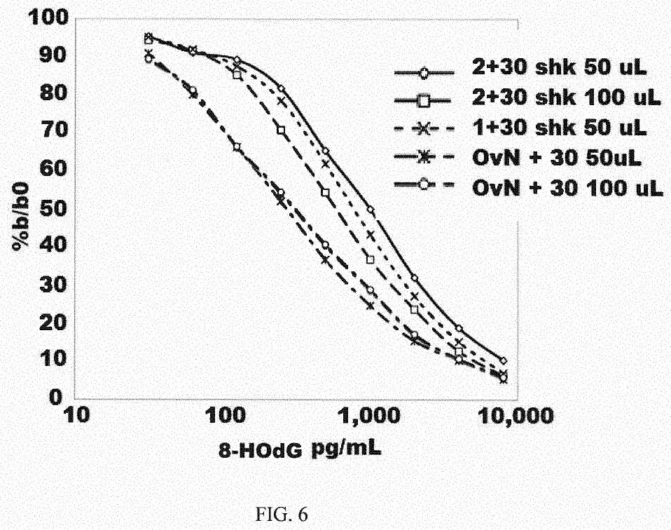

Additionally, the instant methods can be easily adapted to varying assay concentration ranges. Typical variations to commercially available assays for measuring small molecule analytes, e.g., a guanine derivative representing oxidative DNA damage, involve coating the solid phase supports with diminishing amounts of the target analyte. Each solid phase support coating process can take several days to a week to complete. The variations depicted in FIG. 6, which involve different standard ranges, can all be accomplished in one experiment lasting only a few hours. As shown in FIG. 6, the 50% binding point of the 5 curves varies by almost a factor of ten, demonstrating the ease of adapting the instant method to varying assay concentration ranges.

The reagents and devices described herein and used in the practice of the method of this invention can be supplied as individually packaged components of a test kit. Such kits contain a first component including a receptor as described above, immobilized to a solid phase support or not; a second component including a ligand for said receptor, said ligand conjugated to a small molecule analyte as described above, the second component immobilized to the immobilized first component on the solid phase support or not; and a third component including a detection molecule conjugated to a label, the detection molecule capable of specific binding with the small molecule analyte as described above. According to aspects, the test kits include one, two or all of these reagents as well as suitable reagents for providing a colorimetric, fluorometric or chemiluminescent signal from the label, control reagents such as a known amount of the small molecule analyte to be detected for use in producing a standard curve, wash solutions, disposable test devices, filtration devices, and/or instructions for carrying out the method of the invention,

According to aspects, the kits contain a first antibody, protein A or protein G immobilized on a solid phase support; a second antibody conjugated to one or more of 8-hydroxyguanosine, 8-hydroxyguanosine and 8-hydroxy-2′-deoxyguanosine, wherein the first antibody, protein A or protein G is capable of specific binding with the second antibody and wherein the second antibody is immobilized on the solid phase support by specific binding to the first antibody, protein A or protein a Further included is a third component including a monoclonal antibody Fab fragment conjugated to a label, the monoclonal antibody Fab fragment capable of specific binding with one or more of: 8-hydroxyguanosine, 8-hydroxyguanosine and 8-hydroxy-2′-deoxyguanosine According to aspects, the test kits include one, two or all of these reagents as well as suitable reagents for providing a colorimetric, fluorometric or chemiluminescent signal from the label, control reagents such as a known amount of one or more of: 8-hydroxyguanosine, 8-hydroxyguanosine and 8-hydroxy-2′-deoxyguanosine for use in producing a standard curve, wash solutions, disposable test devices, filtration devices, and instructions for carrying out the method of the invention.

According to aspects, the kits contain a first antibody immobilized on a solid phase support wherein the antibody is capable of specific binding to a protein; the protein to which the antibody can specifically bind is conjugated to one or more of 8-hydroxyguanosine, 8-hydroxyguanosine and 8-hydroxy-2′-deoxyguanosine, wherein the protein is optionally immobilized on the solid phase support by specific binding to the antibody or supplied as a reagent for later immobilization on the solid phase support by specific binding, to the antibody. The protein conjugated to one or more of 8-hydroxyguanosine, 8-hydroxyguanosine and 8-hydroxy-2′-deoxyguanosine can be any protein capable of inducing an immune response such that, an antibody can be generated for specific binding to the protein. Non-limiting examples of such proteins include an immunoglobulin or albumin, such as bovine serum albumin and ovalbumin. Further included is a third component including a monoclonal antibody conjugated to a label, the monoclonal antibody capable of specific binding with one or more of: 8-hydroxyguanosine, 8-hydroxyguanosine and 8-hydroxy-2′-deoxyguanosine. According to aspects, the test kits include one, two or all of these reagents as well as suitable reagents for providing a colorimetric, fluorometric or chemiluminescent signal from the label, control reagents such as a known amount of one or more of: 8-hydroxyguanosine, 8-hydroxyguanosine and 8-hydroxy-2′-deoxyguanosine for use in producing a standard curve, wash solutions, disposable test devices, filtration devices, and instructions for carrying out the method of the invention.

According to aspects, the kits contain streptavidin or avidin immobilized on a solid phase support wherein the streptavidin or avidin is capable of specific binding to a corresponding biotin conjugated to a protein, wherein the protein is also conjugated to one or more of 8-hydroxyguanosine, 8-hydroxyguanosine and 8-hydroxy-2′-deoxyguanosine, and wherein the protein is optionally immobilized on the solid phase support by specific binding to the streptavidin or avidin or supplied as a reagent for later immobilization on the solid phase support by specific binding to the streptavidin or avidin. The protein conjugated to one or more of 8-hydroxyguanosine. 8-hydroxyguanosine and 8-hydroxy-2′-deoxyguanosine can be any protein. Non-limiting examples of such proteins include albumin, such as bovine serum albumin and ovalbumin. Further included is a third component including a monoclonal antibody conjugated to a label, the monoclonal antibody capable of specific binding with one or more of: 8-hydroxyguanosine, 8-hydroxyguanosine and 8-hydroxy-2′-deoxyguanosine. According to aspects, the test kits include one, two or all of these reagents as well as suitable reagents for providing a colorimetric, fluorometric or chemiluminescent signal from the label, control reagents such as a known amount of one or more of: 8-hydroxyguanosine, 8-hydroxyguanosine and 8-hydroxy-2′-deoxyguanosine for use in producing a standard curve, wash solutions, disposable test devices, filtration devices, and instructions for carrying, out the method of the invention.

According to aspects, the kits contain biotin immobilized on a solid phase support wherein the biotin is capable of specific binding to a corresponding avidin or streptavidin conjugated to one or more of 8-hydroxyguanosine, 8-hydroxyguanosine and 8-hydroxy-2′-deoxyguanosine. Further included is a third component including a monoclonal antibody conjugated to a label, the monoclonal antibody capable of specific binding with one or more of: 8-hydroxyguanosine, 8-hydroxyguanosine and 8-hydroxy-2′-deoxyguanosine. According to aspects, the test kits include one, two or all of these reagents as well as suitable reagents for providing a colorimetric, fluorometric or chemiluminescent signal from the label, control reagents such as a known amount of one or more of: 8-hydroxyguanosine, 8-hydroxyguanosine and 8-hydroxy-2′-deoxyguanosine for use in producing a standard curve, wash solutions, disposable test devices, filtration devices, and instructions for carrying out the method of the invention.

According to aspects, the kits contain biotin immobilized on a solid phase support wherein the biotin is capable of specific binding to a corresponding avidin or streptavidin conjugated to a protein, wherein the protein is also conjugated to one or more of 8-hydroxyguanosine, 8-hydroxyguanosine and 8-hydroxy-2′-deoxyguanosine, and wherein the protein is optionally immobilized on the solid phase support by specific binding to the biotin or supplied as a reagent for later immobilization on the solid phase support by specific binding to the biotin. The protein conjugated to one or more of 8-hydroxypanosine, 8-hydroxyguanosine and 8-hydroxy-2′-deoxyguanosine can be any protein. Non-limiting examples of such proteins include albumin, such as bovine serum albumin and ovalbumin. Further included is a third component including a monoclonal antibody conjugated to a label, the monoclonal antibody capable of specific binding with one or more of: 8-hydroxyguanosine, 8-hydroxyguanosine and 8-hydroxy-2′-deoxyguanosine. According to aspects, the test kits include one, two or all of these reagents as well as suitable reagents for providing a colorimetric, fluorometric or chemiluminescent signal from the label, control reagents such as a known amount of one or more of: 8-hydroxyguanosine, 8-hydroxyguanosine and 8-hydroxy-2′-deoxyguanosine for use in producing a standard curve, wash solutions, disposable test devices, filtration devices, and instructions for carrying out the method of the invention.

Embodiments of inventive compositions and methods are illustrated in the following examples. These examples are provided for illustrative purposes and are not considered limitations on the scope of inventive compositions and methods.

### EXAMPLES

### Example 1

Immobilized Antigen Assay

1. Preparation of Periodate Oxidized 8-Hydroxyguanosine

7.86 mg of 8-hydroxyguanosine (CAS:3868-31-3) and 13.02 mg of sodium periodate (CAS:7790-28-5) were dissolved in 200 μL of 0.1M sodium acetate buffer, pH 5.5. The mixture was stirred at room temperature for 1 hour. The resulting periodate oxidized 8-hydroxyguanosine (I) was used without purification.

2. Preparation of Periodate Oxidized 8-Hydroxyguanosine:BSA Conjugate

27.64 mg of bovine serum albumin (BSA) (Lampire Biologicals, catalog number 7500802) was mixed with 2.764 mL of 0.1M sodium acetate buffer, pH 5.5 and stirred to dissolve.

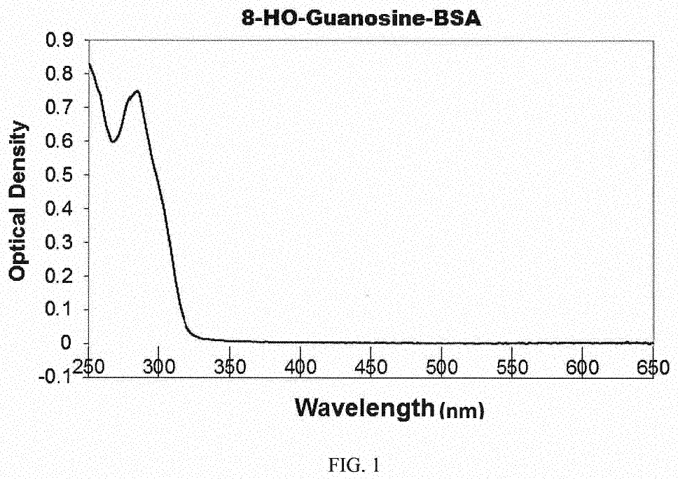

87.7 μL of periodate oxidized 8-hydroxyguanosine and 5 mg of BSA were mixed in 0.1M sodium acetate buffer, pH 5.5, and stirred at room temperature for 30 minutes. The mixture was passed over a BioRad 10DG column equilibrated in 0.1M phosphate buffer, pH 7.2. 1 mL fractions were collected and optical density measured. Those fractions with detectable optical density at 280 nm indicative of presence of 8-hydroxyguanosine:BSA conjugate, were pooled. The 8-hydroxyguanosine:BSA conjugate (II) was stored at −20° C. UV/visible spectrum of the 8-hydroxyguanosine:BSA conjugate is shown in FIG. 1.

3. Coating of Plates With 8-Hydroxyguanosine:BSA Conjugate

8-hydroxyguanosine:BSA conjugate (II) was diluted in PBS at varying concentrations from 10 μg/mL, to 0.01 μg/mL and 150 μL of each concentration was added to each well of high binding 96-well polystyrene microliter plates (Corning Costar 9018). These plates have a binding capacity of approximately 400 to 500 ng IgG/cm². The total volume of each well is 360 microliters with a recommended working volume of 75-200 microliters. The wells were incubated with the 8-hydroxyguanosine:BSA conjugate solutions at room temperature overnight, approximately 15 hours. The 8-hydroxyguanosine:BSA conjugate solution was then aspirated out of the wells and 250 μL of blocking buffer was added to each well and incubated overnight, approximately 15 hours. The blocking buffer used consists of PBS with added BSA and detergent. Following incubation in the blocking buffer, the blocking buffer was aspirated out of the wells and the treated plates were dried in drying cabinets with silica gel over 2 days. The resulting 8-hydroxyguanosine:BSA coated plates (III) were then packaged in metal zip-lock bags with silica gel desiccant and stored at 4° C.

4. Assay Using 8-Hydroxyguanosine:BSA Coated Plates

In this format 8-Hydroxyguanosine:BSA Coated Plates are used as one limited concentration binding partner to samples containing 8-hydroxyguanosine, 8-hydroxyguanosine, and/or 8-hydroxy-deoxyguanosine. Detection of antigens is completed by addition of an antibody that recognizes these compounds specifically. The amount of antibody that is in excess of the antigens in the standard or sample binds to the 8-Hydroxyguanosine:BSA immobilized on the plates.

8-Hydroxy-deoxyguanosine (8HOdG, CAS #88847-89-6) was used as a standard and was dissolved in Assay Buffer. Assay Buffer (AB) contains Tris buffer salts, BSA and detergent. Various RNA/DNA Damage antibodies were tested. All were diluted in Assay Buffer for testing.

50 μL of samples putatively containing 8-hydroxyguanosine or standards containing known amounts of 8HOdG were added to the 8-hydroxyguanosine:BSA coated plates (III), followed by 25 μL of dilutions of specific primary antibodies in Assay Buffer for DNA damage products forming a reaction mixture in the wells. The plate was shaken for 2 hours at room temperature to allow the competitive binding reaction to take place.

At the end of this primary antibody incubation, the wells of the plate were washed with 300 μL Wash Buffer four times. Wash Buffer contains PBS with Tween 20 as the detergent. 100 μL of a goat antibody to IgG labeled with peroxidase, Sigma A9044, at 25 ng/mL was then added. The plate was again shaken for 30 minutes at room temperature to allow binding of the secondary antibody to any primary antibody in the wells.

The well of the plate were then washed with four times with 300 μL of Wash Buffer, followed by addition of 100 μL of chromogenic peroxidase substrate 3,3′,5,5′-tetramethylbenzidine (TMB) (Arbor Assays, catalog number X019) was added to each well and incubated at room temperature for 30 minutes, 50 μL of 1M hydrochloric acid stop solution (Arbor Assays, catalog number X020) was added to each well to stop the color reaction. The optical density of the solution in each well of the plate was determined at 450 nm in a microplate spectrophotometer.

Results are shown in FIG. 2 and illustrate the change of bound peroxidase signal with 1000 ng/mL, 750 ng/mL or 500 ng/mL, of mouse primary antibody specific for 8-hydroxyguanosine.

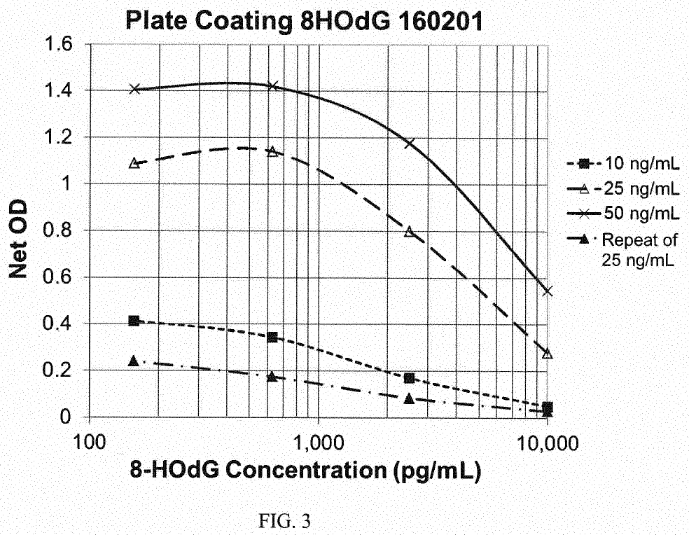

Results of this assay format are also shown in FIG. 3 and demonstrate the lack of reproducibility of this assay format to measure 8-hydroxyguanosine using a microtiter plate coated with varying concentrations of 8-hydroxyguanosine:BSA. FIG. 3 shows that the bound peroxidase signal is very different even when coated with identical amounts of 8-hydroxyguanosine:BSA (25 ng/mL, compare filled and empty triangles).

Further, the bound peroxidase signal is higher when using a lower concentration of antibody (10 ng/mL, filled squares) than the signal observed when 25 ng/mL was used in one assay (25 ng/mL, filled triangle) indicating a lack of reproducibility of the plate coating process using 8-hydroxyguanosine:BSA conjugate at low protein concentrations. Solid squares are with plates coated at 10 ng/mL 8-hydroxyguanosine:BSA conjugate, open triangles 25 ng/mL 8-hydroxyguanosine:BSA conjugate, and crosses are with 50 ng/mL 8-hydroxyguanosine:BSA conjugate. Filled triangles are a previous coating of 25 ng/mL 8-hydroxyguanosine: BSA conjugate.

### Example 2

Example of Analyte Assay of the Present Invention

5. Preparation of Periodate Oxidized 8-Hydroxyguanosine:Rabbit IgG Conjugate

Rabbit IgG (Sigma, catalog number i5006) at 0.848 mg/mL in carbonate buffer at pH 9.4 was treated with periodate oxidized 8-hydroxyguanosine in sodium acetate buffer as outlined in 2. above. The resulting column purified 8-hydroxyguanosine:rabbit IgG (RIgG-8HOG) conjugate (IV) was stored at −20° C.

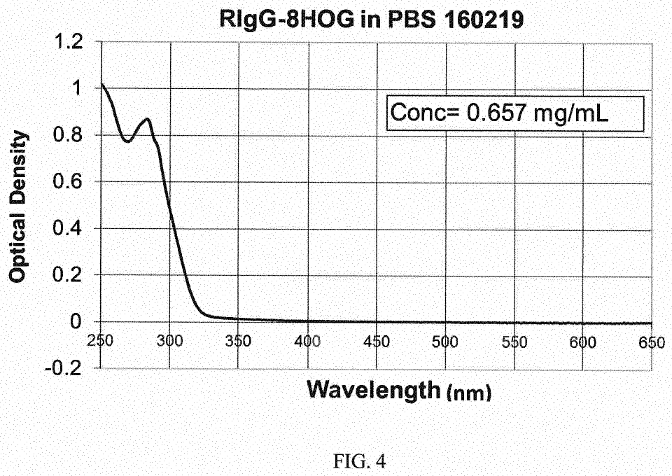

FIG. 4 shows the spectra of the 8-hydroxyguanosine:rabbit IgG conjugate.

6. Peroxidase Labeling of Anti-8-Hydroxyguanosine Antibody

A peroxidase labeling kit (Pierce EZ-Link Activated Peroxidase Labeling kit, catalog number 31497) was used to label DNA damage antibodies including anti-8 hydroxyguanosine mouse monoclonal antibody 7D7E4, anti-8 hydroxyguanosine mouse monoclonal antibody N45.1, anti-8 hydroxyguanosine antibody mouse monoclonal 15A3 (all commercially available from Abeam, PLC, Cambridge, Mass.), following the kit manufacturer's instructions to produce labeled DNA damage antibody (V),

7. Preparation of Secondary Antibody Coated Plates

Plates are coated with goat antibody to rabbit IgG (goat anti-rabbit IgG, Arbor Assays, catalog number A009-10MG) by placing 150 μL of a solution containing 10μg/mL goat anti-rabbit IgG in low ionic strength PBS in each well and incubating at room temperature overnight.

Unbound anti-rabbit IgG is aspirated off the following morning after which 250 μLof Blocking Buffer is added. Blocking Buffer contains 0.2% BSA in PBS. The plates are again incubated overnight at room temperature. The following morning the plates are aspirated and dried. Once dry the plates are stable for many months at 4° C. These goat anti-rabbit IgG coated plates can also be purchased ready to use from various manufacturers such as Arbor Assays, X016-1EA.

8. Assay Using 8-Hydroxyguanosine:Rabbit IgG Conjugate

50 μL of samples putatively containing 8-hydroxyguanosine, 8-hydroxyguanosine, and/or 8-hydroxy-deoxyguanosine or standards containing known amounts of 8HOdG are pipetted into the appropriate wells of the goat anti-rabbit IgG coated plate, X016-1EA. 25 μL of the 8-hydroxyguanosine:rabbit IgG conjugate (IV), (10 ng/mL in Assay:Buffer) are added to each well, followed by 25 μL of the peroxidase labeled DNA damage antibody (V) to initiate the assay. The plate was then shaken for 2 hours at room temperature.

Following the reaction, each well was washed four times with 300 μL of Wash Buffer and 100 μL of TMB added. The plate was incubated at room temperature for 30 minutes. 50 μL of 1M hydrochloric acid stop solution was added to stop the color reaction. The optical density of the solution in each well of the plate was determined at 450 nm in a microplate spectrophotometer.

The results shown in FIG. 5 demonstrate superior characteristics of an inventive assay compared to the standard assay described in Example 1. In this example the labeled DNA Damage antibody concentration is varied by changing the concentration in assay buffer.

50 μL or 100 μL of samples putatively containing 8-hydroxyguanosine, 8-hydroxyguanosine, and/or 8-hydroxy-deoxyguanosine or standards containing known amounts of 8HOdG are pipetted into the appropriate wells of the microtiter plate. 25 μL of the 8-hydroxyguanosine:rabbit IgG conjugate (IV), (10 ng/mL in Assay Buffer) are added to each well, followed by 25 μL of the peroxidase labeled DNA damage antibody (V) to initiate the assay. The plate was then shaken for either 1 hour or 2 hours at room temperature, or was incubated without shaking at 4° C. overnight.

Following the reaction, each well was washed four times with 300 μL of Wash Buffer and 100 μL of TMB added. The plate was incubated at room temperature for 30 minutes, 50 μL of 1M hydrochloric acid stop solution was added to stop the color reaction. The optical density of the solution in each well of the plate was determined at 450 nm in a microplate spectrophotometer.

FIG. 6 shows results of format variations using identical reagents and plates according to aspects of the present invention. In FIG. 6 the following key has been used:

“2+30 shk 50 μL” indicates a 2 hour primary incubation, followed by a 30 minute TMB substrate reaction using 50 μL of sample or standards.

“2+30 shk 100 μL” indicates a 2 hour primary incubation, followed by a 30 minute TMB substrate reaction using 100 μL of sample or standards.

“1+30 shk 50 μL” indicates a 1 hour primary incubation, followed by a 30 minute TMB substrate reaction using 50 μL of sample or standards.

“OvN+30 50 μL” indicates an overnight primary incubation at 4° C. without shaking using 50 μL of sample or standards, followed by a 30 minute TMB substrate reaction.

“OvN+30 100 μL” indicates an overnight primary incubation at 4° C. without shaking using 100 μL of sample or standards, followed by a 30 minute TMB substrate reaction.

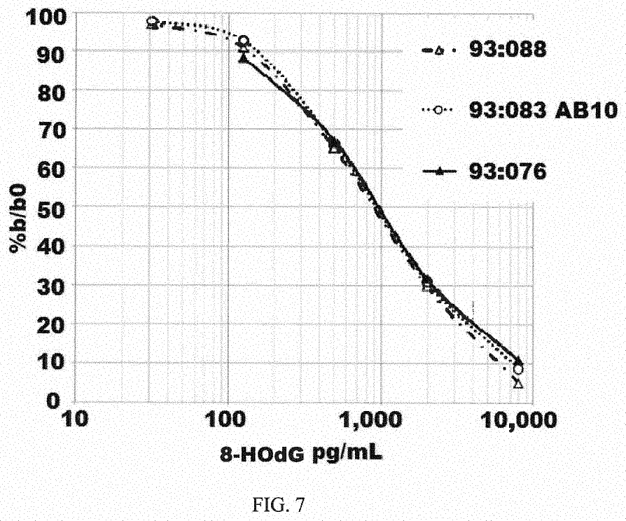

FIG. 7 shows reproducibility of the assay.

The reagents and plates were identical for the various assays shown, but the standard curves were run over a period of over a month with all components being stored at 4° C.

25-100 μL of standards containing known amounts of 8HOdG are pipetted into the appropriate wells of the microtiter plate. 25 μL of the 8-hydroxyguanosine:rabbit IgG conjugate (IV), 10 ng/mL in Assay Buffer, are added to each well, followed by 25 μL of the peroxidase labeled DNA damage antibody (V) to initiate the assay. The plate was then shaken for 2 hours at room temperature.

Following the reaction, each well was washed four times with 300 μL of Wash Buffer and 100 μL of TMB added. The plate was incubated at room temperature for 30 minutes. 50μL. of 1M hydrochloric acid stop solution was added to stop the color reaction. The optical density of the solution in each well of the plate was determined at 450 nm in a microplate spectrophotometer.

25-100 μL of standards containing known amounts of 8HOdG are pipetted into the appropriate wells of the microtiter plate. 25 of the 8-hydroxyguanosine/rabbit IgG conjugate (IV), 10 ng/mL in Assay Buffer, are added to each well, followed by 25 μL of the peroxidase labeled DNA damage antibody (V) to initiate the assay. The plate was then incubated at 4° C. overnight.

Following the reaction, each well was washed four times with 300 μL of Wash Buffer and 100 μL of TMB added. The plate was incubated at room temperature for 30 minutes. 50 of 1M hydrochloric acid stop solution was added to stop the color reaction. The optical density of the solution in each well of the plate was determined at 450 nm in a microplate spectrophotometer.

Graph marked as 93:076 (solid triangles) in FIG. 7 was run on Apr. 1, 2016. Run 93:088 (open triangles) was run on May 2, 2016, None of the reagents are stored freeze dried or below 4°C.

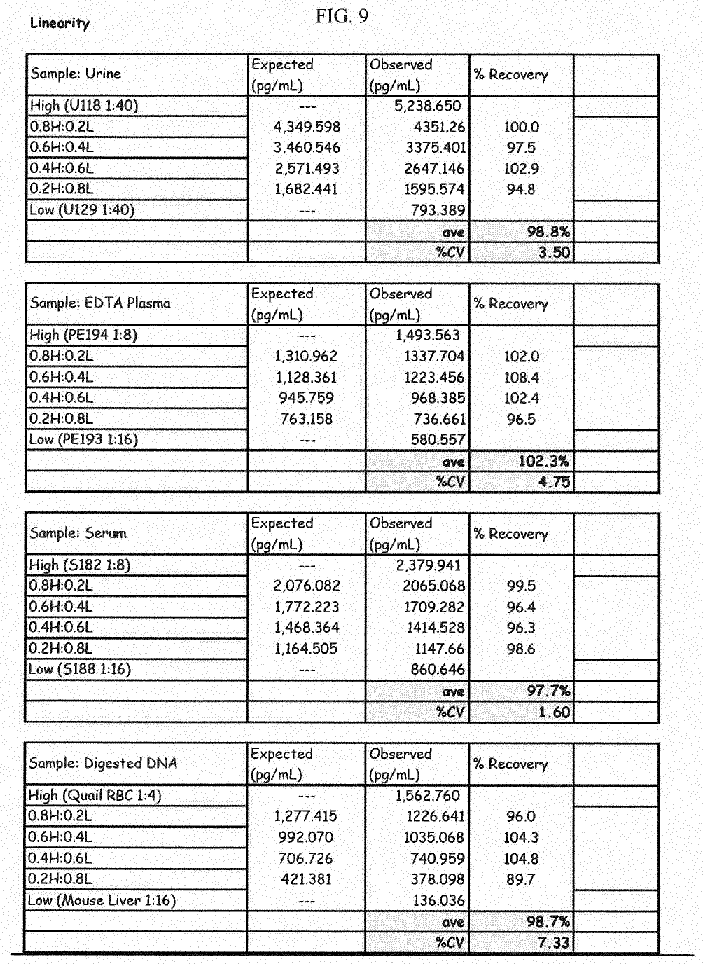

Linearity of the assay is shown in FIG. 9, recoveries are all in the range of 97.7% -102.3%.

### Example 3

Example of Analyte Assay of the Present Invention

9. Activation and Coupling of Cortisol to Sheep IgG

Cortisol-3-carboxymethyloxime (CMO), Sigma, H6635, activated in dry dimethylformamide by treatment with N-hydroxysuccinimide and dicyclohexylcarbodimide to form the cortisol-3-carboxy-N-hydroxysuccinimidyl ester. Activated cortisol-3-CMO added to sheep IgG, Sigma I5131, in borate buffer, pH 8.5 and purified from excess cortisol-3-CMO by dialysis or column chromatography.

10. HRP Labeled Cortisol Antibody

Chicken antibody to cortisol, abeam ab157422, is conjugated with HRP using Pierce EZ-Link Activated Peroxidase Labeling kit, catalog number 31497.

11. Cortisol Assay of the Present Invention

Plates coated with excess donkey anti-sheep IgG antibody would be used, A cortisol-sheep IgG conjugate, HRP-chicken anti-cortisol antibody and standards or samples containing cortisol diluted in a buffer between pH 5 and 9, containing carrier proteins and/or detergents, would be mixed and incubated. After incubation and washing TM B would be added to develop color.

The HRP-chicken anti-cortisol antibody would be in limiting amounts and would bind either cortisol in the samples or the cortisol labeled onto sheep IgG. The amount of HRP-chicken anti-cortisol antibody bound to the cortisol-sheep IgG conjugate would be inversely proportional to the amount of sample or standard cortisol in the assay. The carrier, the cortisol sheep IgG conjugate with bound HRP-chicken anti-cortisol antibody, is captured by the excess donkey anti-sheep IgG on the solid phase. Results similar to those obtained in Example 2 are expected.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method for detecting a small molecule analyte in a sample, comprising:
providing a first component comprising a receptor, wherein the first component is immobilized on a solid phase support, and wherein the receptor does not specifically bind to the small molecule analyte;
providing a second component comprising a ligand for said receptor, said ligand conjugated to a conjugated, small molecule analyte;
providing a third component comprising a detection molecule conjugated to a label, the detection molecule capable of specific binding with the small molecule analyte, wherein said third component does not specifically bind to the first component;
contacting the second component and the immobilized first component under specific binding reaction conditions, whereby the second component is immobilized on the solid phase support by specific binding of the receptor and the ligand, producing an immobilized second component;
contacting a fluid sample containing or suspected of containing the small molecule analyte and the immobilized second component with the third component under specific binding reaction conditions, whereby a labeled immobilized complex is formed between the third component and the immobilized second component by the binding between the detection molecule and the conjugated small molecule analyte, and whereby a labeled non-immobilized complex is formed between the third component and the small molecule analyte of the fluid sample by specific binding between the detection molecule and the small molecule analyte of the fluid sample;
separating the immobilized complex from the non-immobilized complex; and
detecting a signal from the labeled immobilized complex, the labeled non-immobilized complex, or both the labeled immobilized complex and the labeled non-immobilized complex, thereby detecting the small molecule analyte in the sample.

2. The method of claim 1, wherein the small molecule analyte is selected from the group consisting of 8-hydroxyguanosine, 8-hydroxyguanosine, 8-hydroxy-2′-deoxyguanosine, and two or more thereof.

3. The method of claim 1, wherein the receptor is an antibody, a lectin, fibronectin, protein A, protein G, avidin, steptavidin, or two or more thereof.

4. The method of claim 1, wherein the ligand comprises art antibody, a protein, a sugar, biotin a biotin labeled protein, or two or more thereof.

5. The method of claim 1, wherein the fluid sample is saliva, urine, plasma, serum, isolated DNA, isolated RNA, a cell lysate, a tissue sample lysate, cell culture medium, hair, feathers, a fecal sample, cerebrospinal fluid (CSF), milk, or two or more thereof.

6. The method of claim 1, wherein the detection molecule is an antibody specific for the small molecule analyte.

7. The method of claim 1, wherein contacting a fluid sample containing or suspected of containing the small molecule analyte with the immobilized second component is performed concurrently with contacting a fluid sample containing or suspected of containing the small molecule analyte with the third component.

8. The method of claim 1, further comprising contacting the fluid sample with the first component, wherein the fluid sample is contacted with the first component prior to contacting the first component with the second component.

9. The method of claim 1, further comprising quantitating the small molecule analyte in the sample, thereby determining an amount and/or concentration of the small molecule analyte in the sample.

10. The method of claim 9, wherein quantitating the small molecule analyte in the sample comprises comparing the signal from the labeled immobilized complex, the labeled non-immobilized complex, or both the labeled immobilized complex and the labeled non-immobilized complex with a standard, the standard generated using a known amount of the small molecule analyte.

11. The method of claim 10, wherein the standard is a standard curve.

12. A method for detecting 8-hydroxy-2′-deoxyguanosine (8-HOdG) in a sample, comprising:
providing a first component comprising a receptor, wherein the first component is immobilized on a solid phase support, and the receptor does not specifically bind 8-HOdG;
providing a second component comprising a ligand for said receptor, said ligand conjugated to 8-HOdG such that the second component comprises ligand-conjugated 8-HOdG;
providing a third component comprising a detection molecule conjugated to a label, the detection molecule capable of specific binding with 8-HOdG, wherein the third component does not specifically bind to the first component;
contacting: 1) a fluid sample containing or suspected of containing 8-HOdG and 2) the immobilized first component;
contacting: 1) the second component and 2) the immobilized first component under specific binding reaction conditions, whereby the second component is immobilized on the solid phase support by specific binding of the receptor and the ligand, thereby producing an immobilized second component;
contacting a fluid sample and immobilized second component with the third component under specific binding reaction conditions, whereby an immobilized complex is formed between the third component and the immobilized second component by specific binding between the detection molecule and the ligand-conjugated 8-HOdG, and whereby a non-immobilized complex is formed between the third component and the 8-HOdG of the fluid sample by specific binding between the detection molecule and the 8-HOdG of the fluid sample;
separating the immobilized complex from the non-immobilized complex; and
detecting the label of the immobilized complex,

13. The method of claim 12, wherein:
the receptor is an antibody;
the ligand is an antibody;
the detection molecule is an antibody;
the label is an enzyme, a peroxidase, or horseradish peroxidase; or
the method further comprises contacting the label with a substrate and detecting the label comprises detecting either the substrate or a product of the substrate

14. The method of claim 12, further comprising quantitating the 8-HOdG in the sample, thereby determining an amount and/or concentration of the 8-HOdG in the sample,
